SEQUENCE LISTING (10 sequences)

SEQ ID NO: 1 - protein, 446 aa, Homo sapiens
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 116 aa, Homo sapiens
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSS

SEQ ID NO: 3 - protein, 6 aa, Homo sapiens
SDFAWN

SEQ ID NO: 4 - protein, 16 aa, Homo sapiens
YISYSGNTRYQPSLKS

SEQ ID NO: 5 - protein, 9 aa, Homo sapiens
VTAGRGFPY

SEQ ID NO: 6 - protein, 214 aa, Homo sapiens
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 107 aa, Homo sapiens
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIK

SEQ ID NO: 8 - protein, 11 aa, Homo sapiens
HSSQDINSNIG

SEQ ID NO: 9 - protein, 7 aa, Homo sapiens
HGTNLDD

SEQ ID NO: 10 - protein, 9 aa, Homo sapiens
VQYAQFPWT

(19) United States
(12) Patent Application Publication
(10) Pub. No.: US 2020/0368525 A1
(43) Pub. Date: Nov. 26, 2020

(54) Methods of Treating Glioblastoma

(71) Applicants: Abbvie Inc., North Chicago, IL (US); Novocure GmbH, Root D4 (CH)

(72) Inventors: David MAAG, Green Oaks, IL (US); Moshe GILADI, Moshav Herut (IL); Rosa S. SCHNAIDERMAN, Haifa (IL); Einav ZEEVI, Zichron Yaakov (IL); Eilon KIRSON, Ramat Hasharon (IL)

(21) Appl. No.: 16/764,248

(22) PCT Filed: Nov. 19, 2018
(86) PCT No.: PCT/US2018/061846

§ 371 Date: May 14, 2020

Related U.S. Application Data
(60) Provisional application No. 62/587,830, filed on Nov. 17, 2017.

(51) Int. Cl. A61N 1/36; A61P 35/00; A61N 1/04; A61K 47/68; C07K 16/28

(52) U.S. Cl. CPC A61N 1/36002; A61P 35/00; A61N 1/0428; A61K 2039/505; C07K 16/2818; A61N 1/0412; A61K 47/6849

(57) ABSTRACT

The present disclosure provides a method for treating cancer in a patient harboring an EGFR-expressing tumor and/or tumor cell, such as glioblastoma, comprising the combination of (i) applying an AC electric field to a target area, wherein the target area comprises an EGFR-expressing tumor or cancer cell); and (ii) administering an effective amount of depatuxizumab mafodotin.

### CROSS-REFERENCE TO RELATED APPLICATIONS

This application claims benefit to U.S. provisional application Ser. No. 562/587,830, filed Nov. 17, 2017, under 35 U.S.C. § 119(e). The entire teachings of the referenced application are incorporated herein by reference in their entirety.

### FIELD

The present disclosure features methods of treating cancer, in particular glioblastoma, using a combination of depatuxizumab mafodotin and Tumor Treating Fields.

### BACKGROUND

Antibody-drug conjugates (ADCs) are a rapidly expanding class of cancer therapy that combine the targeting specificity of monoclonal antibodies (mAbs) with the cytotoxicity of potent small molecules. A distinct clinical advantage of ADCs is their ability to deliver toxic payloads directly to a tumor, bypassing downstream resistance mechanisms related to intracellular signaling.

Tumor Treating Fields, or TTFields, are low intensity (e.g., 1-3 V/cm), alternating electric fields within the intermediate frequency range (100-300 kHz). This non-invasive treatment targets solid tumors and is described, for example, in U.S. Pat. No. 7,565,205, which is incorporated by reference herein in its entirety. TTFields disrupt cell division through physical interactions that interfere with the assembly of key molecules needed for mitosis. TTFields therapy is an approved mono-treatment for recurrent glioblastoma, and an approved combination therapy with chemotherapy for newly diagnosed patients. These electric fields are induced non-invasively by transducer arrays (i.e., arrays of electrodes) placed directly onto the patient's scalp. TTFields also appear to be beneficial for treating tumors in other parts of the body.

TTFields are established as an anti-mitotic cancer treatment modality because they interfere with proper microtubule assembly during the metaphase portion of the cell cycle which eventually leads to the destruction of the cells during the telophase and cytokinesis portions of the cell cycle. For cancer treatment, non-invasive devices were developed with capacitively-coupled transducers that are placed directly onto the skin region closest to the tumor. The efficacy increases with increasing field strength and the optimal frequency is specific to the cancer cell type, with 200 kHz being the TTFields frequency for which inhibition of glioma cells has been shown to be the highest. For patients with glioblastoma multiforme (GBM), the device for delivering TTFields therapy is called Optune™.

Despite the availability of Tumor Treating Field-based therapies, glioblastoma multiforme (GBM) continues to be the most common and aggressive primary malignancy of the central nervous system in adults. Accordingly, there remains a need in the art for effective methods of treating glioblastoma.

### SUMMARY

In embodiments, the present disclosure provides methods for treating cancer in patients that harbor EGFR-expressing tumors, the method comprising a combination of (i) applying an electric field to a target area (where the target area comprises an EGFR-expressing tumor or cancer cell); and (ii) administering an effective amount of depatuxizumab mafodotin to said patient. In embodiments, the cancer expresses the mutant EGFRvIII. In embodiments, the cancer is glioblastoma.

In embodiments, the present disclosure provides methods for inhibiting the growth of EGFR-expressing tumors, the method comprising a combination of (i) applying an electric field to a target area (where the target area includes an EGFR-expressing tumor or cancer cell); and (ii) administering an effective amount of depatuxizumab mafodotin.

### DESCRIPTION OF THE DRAWINGS

FIGS. 1A and 1B show the efficacy of the combined treatment of TTFields and depatuxizumab mafodotin in U87MG glioma cells. U87 MG glioma cells grown in various depatuxizumab mafodotin concentrations were treated with TTFields (200 kHz, 1.6 V/cm RMS) for 72 hours. In FIG. 1A, the number of cells was determined at the end of treatment and is expressed as a percentage of control. The expected number of cells was calculated by multiplying the fraction of surviving cells when TTFields were applied alone with the fraction of surviving cells when depatuxizumab mafodotin was applied alone at each concentration. As shown, the combined treatment of TTFields and depatuxizumab mafodotin (denoted as “ABT-414”) led to a significant reduction in the number of U87-MG cells (P<0.001) compared to each treatment alone at all drug concentrations except 80 nM, whereas the combination with the control Ab095-MMAF ADC (i.e., an MMAF-based antibody conjugate that targets tetanus toxoid, denoted as “ADC”) did not. In FIG. 1B, induction of apoptosis was tested using flow cytometry (7AAD−Annexin V−(live cells), 7AAD+/Annexin V+(late apoptosis), 7AAD−/Annexin V+ (early apoptosis), 7AAD+/annexin V−). As shown, a rapid increase in the number of cells undergoing apoptosis (both early and late) was seen for the combination treatment of TTfields and depatuxizumab mafodotin compared to each treatment alone (denoted as “ABT”) in all tested concentrations, whereas the combination with the control Ab095-MMAF ADC (i.e., an MMAF-based antibody conjugate that targets tetanus toxoid, denoted as “ADC”) did not.

FIGS. 2A and 2B show the efficacy of the combined treatment of TTFields and depatuxizumab mafodotin in the U87MGde2-7 cells, a glioma cell line expressing the mutant EGFRvIII. U87MGde2-7 glioma cells grown in various depatuxizumab mafodotin concentrations were treated with TTFields (200 kHz, 1.6 V/cm RMS) for 72 hours. In FIG. 2A, the number of cells was determined at the end of treatment and is expressed as a percentage of control. The expected number of cells was calculated by multiplying the fraction of surviving cells when TTFields were applied alone with the fraction of surviving cells when depatuxizumab mafodotin was applied alone at each concentration. As shown, the combined treatment of TTFields and depatuxizumab mafodotin (denoted as “ABT-414”) led to a significant reduction in the number of U87MGde2-7 cells compared to either treatment alone, whereas the combination with the control Ab095-MMAF ADC (i.e., an MMAF-based antibody conjugate that targets tetanus toxoid, denoted as “ADC”) did not. In FIG. 2B, induction of apoptosis was tested using flow cytometry (7AAD−/Annexin V− (live cells), 7AAD+/Annexin V+ (late apoptosis), 8AAD−/Annexin V+ (early apoptosis), 8AAD+/annexin V−). As shown, a rapid increase in the number of cells undergoing apoptosis (both early and late) was seen for the combination treatment of TTFields and depatuxizumab mafodotin (denoted as “ABT”) in all tested concentrations, whereas the combination with the control Ab095-MMAF ADC (i.e., an MMAF-based antibody conjugate that targets tetanus toxoid, denoted as “ADC”) did not.

FIG. 3 is a schematic block diagram of an apparatus for applying an electric field according to one exemplary embodiment for selectively destroying cells.

FIG. 4 is a simplified schematic diagram of an equivalent electric circuit of insulated electrodes of the apparatus of FIG. 3.

FIG. 5 is a cross-sectional illustration of a skin patch incorporating the apparatus and for placement on a skin surface for treating a tumor or the like.

FIG. 6 is a cross-sectional illustration of the insulated electrodes implanted within the body for treating a tumor or the like.

FIG. 7A-7D are cross-sectional illustrations of various constructions of the insulated electrodes of the apparatus of FIG. 3.

### DETAILED DESCRIPTION

### Definitions

In order that the disclosure may be more readily understood, certain terms are first defined. In addition, it should be noted that whenever a range of values of a parameter is recited, it is intended that values and ranges intermediate to the recited values are also intended to be part of the present disclosure.

The terms “treat”, “treating” and “treatment” refer to a method of alleviating or abrogating a disease and/or its attendant symptoms.

The term “subject” is defined to include animals such as mammals, including, but not limited to, primates (e.g., humans), cows, sheep, goats, horses, dogs, cats, rabbits, rats, mice, and the like. In embodiments, the subject is a human.

The terms “patient” and “subject” are used herein interchangeably.

The terms “anti-epidermal growth factor antibody drug conjugate” or “anti-EGFR antibody drug conjugate” and “anti-EGFR ADC”, used interchangeably herein, refer to an antibody-drug conjugate comprising an antibody that specifically binds to EGFR, whereby the antibody is conjugated to a drug, e.g., a cytotoxic agent such as an auristatin (e.g., monomethyl auristatin F). In embodiments, the anti-EGFR antibody is conjugated to MMAF via a maleimidocaproyl (mc) linkage. In embodiments, the anti-EGFR antibody is depatuxizumab mafodotin.

The term “auristatin”, as used herein, refers to a family of antimitotic agents. Auristatin derivatives are also included within the definition of “auristatin.” Examples of auristatins include, for example, auristatin E (AE), monomethylauristatin E (MMAE), monomethylauristatin F (MMAF), and synthetic analogs of dolastatin.

The term “anti-EGFR antibody” refers to an antibody that specifically binds to EGFR. An antibody “which binds” an antigen of interest, e.g. EGFR, is one capable of binding that antigen with sufficient affinity such that the antibody is useful in targeting a cell expressing the antigen.

The term “antibody” broadly refers to an immunoglobulin (Ig) molecule, generally comprised of four polypeptide chains, two heavy (H) chains, and two light (L) chains. Antibodies comprise complementarity determining regions (CDRs), also known as hypervariable regions, in both the light chain and heavy chain variable domains. The more highly conserved portions of the variable domains are called the framework (FR). As is known in the art, the amino acid position/boundary delineating a hypervariable region of an antibody can vary, depending on the context and the various definitions known in the art. Some positions within a variable domain may be viewed as hybrid hypervariable positions in that these positions can be deemed to be within a hypervariable region under one set of criteria, while being deemed to be outside a hypervariable region under a different set of criteria. One or more of these positions can also be found in extended hypervariable regions. The variable domains of native heavy and light chains each comprise four FR regions, largely by adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. See Kabat et al., Sequences of Proteins of Immunological Interest (National Institute of Health, Bethesda, Md. 1987). As used herein, numbering of immunoglobulin amino acid residues is done according to the immunoglobulin amino acid residue numbering system of Kabat et al. unless otherwise indicated.

The term “monoclonal antibody” as used herein is not limited to antibodies produced through hybridoma technology. A monoclonal antibody is derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof. In many uses of the present disclosure, including in vivo use of ADCs including anti EGFR antibodies in humans, chimeric, primatized, humanized, or human antibodies can suitably be used.

“Humanized” forms of non-human (e.g., murine) antibodies are chimeric immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., 1988, Nature 332:323-7; U.S. Pat. Nos. 5,530,101; 5,585,089; 5,693,761; 5,693,762; and 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332, all of which are hereby incorporated by reference in their entireties.

Anti-EGFR ADCs of the present disclosure may comprise full-length (intact) antibody molecules, as well as antigen binding fragments that are capable of specifically binding EGFR. Examples of antibody binding fragments include, by way of example and not limitation, Fab, Fab′, F(ab′)2, Fv fragments, single chain Fv fragments and single domain fragments.

As used herein, the term “effective amount” or “therapeutically effective amount” refers to the amount of a drug (e.g., an ADC such as depatuxizumab mafodotin) which is sufficient to reduce or ameliorate the severity and/or duration of a disorder, e.g., cancer, or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent). The effective amount of an ADC may, for example, inhibit tumor growth (e.g., inhibit an increase in tumor volume), decrease tumor growth (e.g., decrease tumor volume), reduce the number of cancer cells, and/or relieve to some extent one or more of the symptoms associated with the cancer. The effective amount may, for example, improve disease free survival (DFS), improve overall survival (OS), or decrease likelihood of recurrence.

The term “combination” or “combination therapy” refers to the administration of two or more therapies, e.g., depatuxizumab mafodotin and TTFields. The two therapies may be administered concomitantly in which case both therapies are administered together or substantially together, or sequentially in which case one therapy may be administered prior to the other therapy.

The term “EGFR expressing tumor” or “cancer having EGFR expression” refers to a tumor which expresses epidermal growth factor receptor (EGFR) protein. In one embodiment, EGFR expression in a tumor is determined using immunohistochemical staining of tumor cell membranes, where any immunohistochemical staining above background level in a tumor sample indicates that the tumor is an EGFR expressing tumor. Methods for detecting expression of EGFR in a tumor are known in the art, e.g., the EGFR pharmDx™ Kit (Dako). In contrast, an “EGFR negative tumor” is defined as a tumor having an absence of EGFR membrane staining above background in a tumor sample as determined by immunohistochemical techniques.

The term “EGFRvIII positive tumor” or “cancer having EGFRvIII expression” as used herein, refers to a tumor which expresses epidermal growth factor receptor (EGFR) protein containing a specific mutation, referred to as EGFRvIII. In one embodiment, EGFRvIII expression in a tumor is determined using immunohistochemical staining of tumor cell membranes, where any immunohistochemical staining above background level in a tumor sample indicates that the tumor is an EGFRvIII expressing tumor. Methods for detecting expression of EGFR in a tumor are known in the art, and include immunohistochemical assays. In contrast, an “EGFRvIII negative tumor” is defined as a tumor having an absence of EGFRvIII membrane staining above background in a tumor sample as determined by immunohistochemical techniques.

The terms “overexpress,” “overexpression,” or “overexpressed” interchangeably refer to a gene that is transcribed or translated at a detectably greater level, usually in a cancer cell, in comparison to a normal cell. Overexpression therefore refers to both overexpression of protein and RNA (due to increased transcription, post transcriptional processing, translation, post translational processing, altered stability, and altered protein degradation), as well as local overexpression due to altered protein traffic patterns (increased nuclear localization), and augmented functional activity, e.g., as in an increased enzyme hydrolysis of substrate. Thus, overexpression refers to either protein or RNA levels. Overexpression can also be by 50%, 60%, 70%, 80%, 90% or more in comparison to a normal cell or comparison cell. In certain embodiments, the methods described herein are used to treat solid tumors likely to overexpress EGFR.

The term “administering” as used herein is meant to refer to the delivery of a substance (e.g., an anti-EGFR ADC such as depatuxizumab mafodotin) to achieve a therapeutic objective (e.g., the treatment of an EGFR-associated disorder). Modes of administration may be parenteral, enteral, and topical. Parenteral administration is usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Depatuxizumab mafodotin (also referred to as “depatux-m” or “ABT-414”, also abbreviated in the Figures of the present disclosure as “ABT”) is an antibody-drug conjugate (ADC) targeting EGFR. It is composed of an EGFR IgG1 monoclonal antibody (depatuxizumab) conjugated to the tubulin inhibitor monomethyl auristatin F via a stable maleimidocaproyl link. Depatuxizumab mafodotin is being investigated to treat cancer, in particular 1 L and 2 L glioblastoma (GBM) and solid tumors is currently undergoing clinical trials. For example, M12-356 is an open-label study with three escalation and expansion cohorts in which sixty-six patients with EGFR-amplified rGBM were treated with depatux-m at 1.25 mg/kg every two weeks.

The term “TTFields” as used herein is meant to refer to Tumor Treating Fields, and generally refers to the use of alternating electric fields to treat cancer. U.S. Pat. Nos. 6,868,289 and 7,016,725, each of which is incorporated herein by reference in its entirety, disclose methods and apparatuses for treating tumors using AC electric fields in the range of 1-10 V/cm, at frequencies between 50 kHz and 500 kHz, and that the effectiveness of those fields is increased when more than one field direction is used (e.g., when the field is switched between two or three directions that are oriented about 90° apart from each other). For purposes of the present disclosure, the definition of “TTFields” encompasses the use of the OPTUNE® device for the treatment of cancer.

In embodiments, the present disclosure relates to a method for the treatment of a cancer expressing EGFR, wherein the method comprises administering tumor treating fields (TTFields) and an effective amount of depatuxizumab mafodotin. In embodiments, the cancer expresses the mutant EGFRvIII. In embodiments, the cancer is glioblastoma.

In embodiments, the present disclosure relates to a method for the treatment of a cancer in patients that harbor EGFR-expressing tumors, wherein the method comprises the combination of (i) administering an AC electric field to a target area, wherein the target area comprises an EGFR-expressing tumor or cancer cell, and (ii) administering an effective amount of an anti-EGFR antibody conjugated to an auristatin. In embodiments, the auristatin is MMAF. In embodiments, the MMAF is conjugated to the antibody with a maleimidocaproyl linker. In embodiments, the anti-EGFR antibody comprises a heavy chain variable region comprising complementary determining regions (CDRs) comprising the amino acid sequences set forth in SEQ ID Nos: 3, 4, and 5, and a light chain variable region comprising CDRs comprising the amino acid sequences set forth in SEQ ID Nos: 8, 9, and 10. In embodiments, the anti-EGFR antibody comprises a heavy chain variable region comprising the amino acid sequence set forth in SEQ ID NO: 2, and a light chain variable region comprising the amino acid sequence set forth in SEQ ID NO: 7. In embodiments, the anti-EGFR antibody comprises a heavy chain comprising the amino acid sequence set forth in SEQ ID NO: 1, and a light chain comprising the amino acid sequence set forth in SEQ ID NO: 6. In embodiments, the anti-EGFR antibody conjugated to an auristatin is depatuxizumab mafodotin.

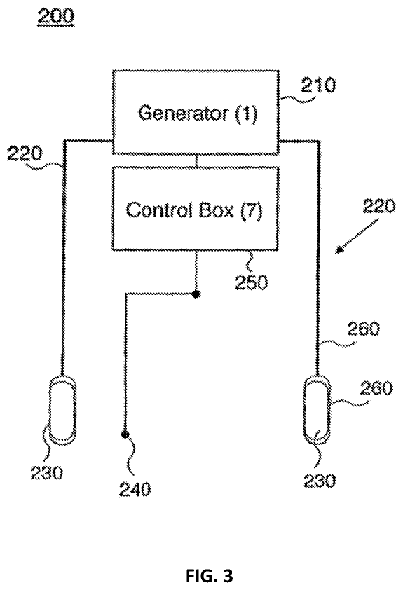

FIG. 3 is an example of an apparatus that is suitable for use in treating live patients with combined TTField and drug therapy (such as an anti-EGFR ADC, e.g., depatuxizumab mafodotin), and it may be used in combination with any conventional drug delivery mechanism (not shown) to implement the combined TTField and drug therapy. FIG. 3 is a simple schematic diagram of the electronic apparatus 200 illustrating the major components thereof The electronic apparatus 200 generates the desired electric waveforms. The apparatus 200 includes a generator 210 and a pair of conductive leads 220 that are attached at one end thereof to the generator 210. The opposite ends of the leads 220 are connected to insulated conductors 230 that are activated by the electric signals (e.g., waveforms). The insulated conductors 230 are also referred to hereinafter as isolects 230. Optionally and according to another embodiment, the apparatus 200 includes a temperature sensor 240 and a control box 250 which are both added to control the amplitude of the electric field generated so as not to generate excessive heating in the area that is treated.

The generator 210 generates an alternating voltage waveform at frequencies in the range from about 50 KHz to about 500 KHz (such as from about 100 KHz to about 300 KHz). The required voltages are such that the electric field intensity in the tissue to be treated is in the range of about 0.1 V/cm to about 10 V/cm, such as between about 1 V/cm and about 5 V/Cm. To achieve this field, the actual potential difference between the two conductors in the isolects 230 is determined by the relative impedances of the system components, as described below.

When the control box 250 is included, it controls the output of the generator 210 so that it will remain constant at the value preset by the user or the control box 250 sets the output at the maximal value that does not cause excessive heating, or the control box 250 issues a warning or the like when the temperature (sensed by temperature sensor 240) exceeds a preset limit.

When the control box 250 is included, it controls the output of the generator 210 so that it will remain constant at the value preset by the user or the control box 250 sets the output at the maximal value that does not cause excessive heating, or the control box 250 issues a warning or the like when the temperature (sensed by temperature sensor 240) exceeds a preset limit.

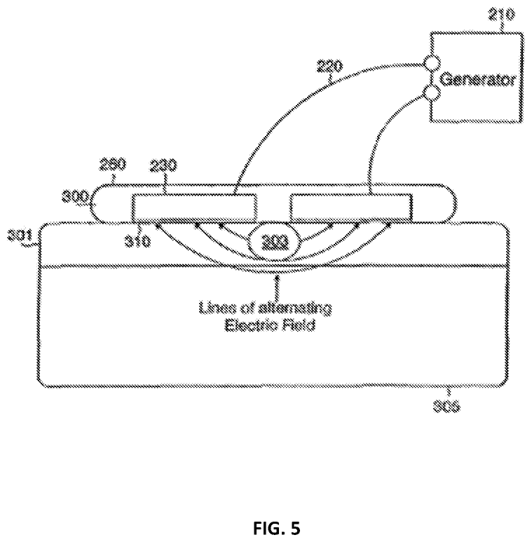

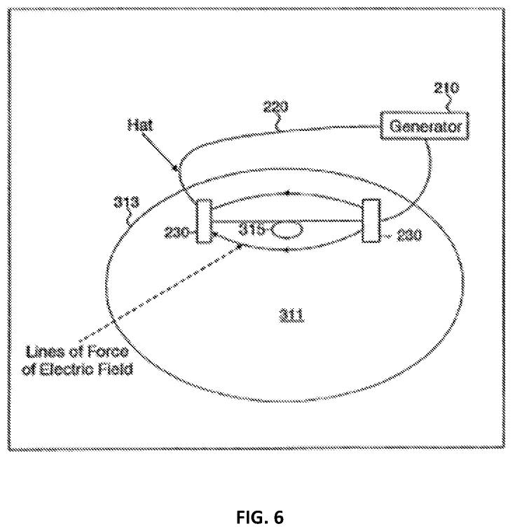

The specifications of the apparatus 200 as a whole and its individual components are largely influenced by the fact that at the frequency of the TTFields (50 KHz-500 KHz), living systems behave according to their “Ohmic”, rather than their dielectric properties. The only elements in the apparatus 200 that behave differently are the insulators of the isolects 230 (see FIGS. 5 and 6). The isolects 200 consist of a conductor in contact with a dielectric that is in contact with the conductive tissue thus forming a capacitor.

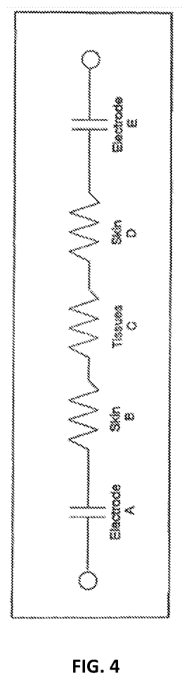

The details of the construction of the isolects 230 is based on their electric behavior that can be understood from their simplified electric circuit when in contact with tissue as generally illustrated in FIG. 4. In the illustrated arrangement, the potential drop or the electric field distribution between the different components is determined by their relative electric impedance, i.e., the fraction of the field on each component is given by the value of its impedance divided by the total circuit impedance. For example, the potential drop on element ΔVA=A/(A+B+C+D+E). Thus, for DC or low frequency AC, practically all the potential drop is on the capacitor (that acts as an insulator). For relatively very high frequencies, the capacitor practically is a short and therefore, practically all the field is distributed in the tissues. At the frequencies of the TTFields (e.g., 50 KHz to 500 KHz), which are intermediate frequencies, the impedance of the capacitance of the capacitors is dominant and determines the field distribution. Therefore, in order to increase the effective voltage drop across the tissues (field intensity), the impedance of the capacitors is to be decreased (i.e., increase their capacitance). This can be achieved by increasing the effective area of the “plates” of the capacitor, decrease the thickness of the dielectric or use a dielectric with high dielectric constant.

In order to optimize the field distribution, the isolects 230 are configured differently depending upon the application in which the isolects 230 are to be used. There are two principle modes for applying the TTFields. First, the TTFields can be applied by external isolects and second, the TTFields can be applied by internal isolects.

TTFields that are applied by external isolects can be of a local type or widely distributed type. The first type includes, for example, the treatment of skin tumors and treatment of lesions close to the skin surface. FIG. 5 illustrates an exemplary embodiment where the isolects 230 are incorporated in a skin patch 300. The skin patch 300 can be a self-adhesive flexible patch with one or more pairs of isolects 230. The patch 300 includes internal insulation 310 (formed of a dielectric material) and the external insulation 260 and is applied to skin surface 301 that contains a tumor 303 either on the skin surface 301 or slightly below the skin surface 301. Tissue is generally indicated at 305. To prevent the potential drop across the internal insulation 310 to dominate the system, the internal insulation 310 must have a relatively high capacity. This can be achieved by a large surface area; however, this may not be desired as it will result in the spread of the field over a large area (e.g., an area larger than required to treat the tumor). Alternatively, the internal insulation 310 can be made very thin and/or the internal insulation 310 can be of a high dielectric constant. As the skin resistance between the electrodes (labeled as A and E in FIG. 4) is normally significantly higher than that of the tissue (labeled as C in FIG. 4) underneath it (1-10 KΩ vs. 0.1-1 KΩ), most of the potential drop beyond the isolects occurs there. To accommodate for these impedances (Z), the characteristics of the internal insulation 310 (labeled as B and D in FIG. 4) should be such that they have impedance preferably under 100 KΩ at the frequencies of the TTFields (e.g., 50 KHz to 500 KHz). For example, if it is desired for the impedance to be about 10 K Ohms or less, such that over 1% of the applied voltage falls on the tissues, for isolects with a surface area of 10 mm², at frequencies of 200 KHz, the capacity should be on the order of 10⁻¹⁰ F., which means that using standard insulations with a dielectric constant of 2-3, the thickness of the insulating layer 310 should be about 50-100 microns. An internal field 10 times stronger would be obtained with insulators with a dielectric constant of about 20-50.

Using an insulating material with a high dielectric constant increases the capacitance of the electrodes, which results in a reduction of the electrodes' impedance to the AC signal that is applied by the generator 1 (shown in FIG. 3). Because the electrodes A, E are wired in series with the target tissue C, as shown in FIG. 4, this reduction in impedance reduces the voltage drop in the electrodes, so that a larger portion of the applied AC voltage appears across the tissue C. Since a larger portion of the voltage appears across the tissue, the voltage that is being applied by the generator 1 can be advantageously lowered for a given field strength in the tissue.

The desired field strength in the tissue being treated may be between about 0.1 V/cm and about 10 V/cm, such as between about 2 V/cm and 3 V/cm or between about 1 V/cm and about 5 V/cm. If the dielectric constant used in the electrode is sufficiently high, the impedance of the electrodes A, E drops down to the same order of magnitude as the series combination of the skin and tissue B, C, D. One example of a suitable material with an extremely high dielectric constant is CaCu₃Ti₄O₁₂, which has a dielectric constant of about 11,000 (measured at 100 kHz). When the dielectric constant is this high, useful fields can be obtained using a generator voltage that is on the order of a few tens of Volts.

Since the thin insulating layer can be very vulnerable, etc., the insulation can be replaced by very high dielectric constant insulating materials, such as titanium dioxide (e.g., rutile), the dielectric constant can reach values of about 200. There are a number of different materials that are suitable for use in the intended application and have high dielectric constants. For example, some materials include: lithium niobate (LiNbO₃), which is a ferroelectric crystal and has a number of applications in optical, pyroelectric and piezoelectric devices; yttrium iron garnet (YIG) is a ferromagnetic crystal and magneto-optical devices, e.g., optical isolator can be realized from this material; barium titanate (BaTiO₃) is a ferromagnetic crystal with a large electro-optic effect; potassium tantalate (KTaO₃) which is a dielectric crystal (ferroelectric at low temperature) and has very low microwave loss and tunability of dielectric constant at low temperature; and lithium tantalate (LiTaO₃) which is a ferroelectric crystal with similar properties as lithium niobate and has utility in electro-optical, pyroelectric and piezoelectric devices. Insulator ceramics with high dielectric constants may also be used, such as a ceramic made of a combination of Lead Magnesium Niobate and Lead Titanate. It will be understood that the aforementioned exemplary materials can be used in combination with the present device where it is desired to use a material having a high dielectric constant.

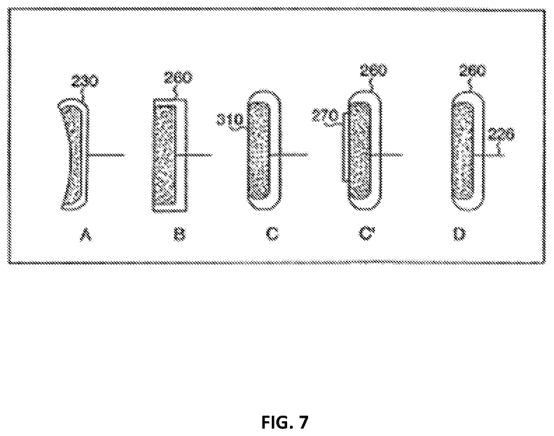

One must also consider another factor that affects the effective capacity of the isolects 230, namely the presence of air between the isolects 230 and the skin. Such presence, which is not easy to prevent, introduces a layer of an insulator with a dielectric constant of 1.0, a factor that significantly lowers the effective capacity of the isolects 230 and neutralizes the advantages of the titanium dioxide (rutile), etc. To overcome this problem, the isolects 230 can be shaped so as to conform with the body structure and/or (2) an intervening filler 270 (as illustrated in FIG. 7C), such as a gel, that has high conductance and a high effective dielectric constant, can be added to the structure. The shaping can be pre-structured (see FIG. 7A) or the system can be made sufficiently flexible so that shaping of the isolects 230 is readily achievable. The gel can be contained in place by having an elevated rim as depicted in FIGS. 7C and 7C′. The gel can be made of hydrogels, gelatins, agar, etc., and can have salts dissolved in it to increase its conductivity. FIGS. 7A-7C illustrate various exemplary configurations for the isolects 230. The exact thickness of the gel is not important so long as it is of sufficient thickness that the gel layer does not dry out during the treatment. In one exemplary embodiment, the thickness of the gel is about 0.5 mm to about 2 mm. Preferably, the gel has high conductivity, is tacky, and is biocompatible for extended periods of time. One suitable gel is AG603 Hydrogel, which is available from AmGel Technologies, 1667 S. Mission Road, Fallbrook, Calif. 92028-4115, USA.

In order to achieve the desirable features of the isolects 230, the dielectric coating of each should be very thin, for example from between 1-50 microns. Since the coating is so thin, the isolects 230 can easily be damaged mechanically or undergo dielectric breakdown. This problem can be overcome by adding a protective feature to the isolect's structure so as to provide desired protection from such damage. Examples of some suitable protective features are described in published application US2005/0209642, which is incorporated herein by reference.

However, the capacity is not the only factor to be considered. The following two factors also influence how the isolects 230 are constructed. The dielectric strength of the internal insulating layer 310 and the dielectric losses that occur when it is subjected to the TTFields, i.e., the amount of heat generated. The dielectric strength of the internal insulation 310 determines at what field intensity the insulation will be “shorted” and cease to act as an intact insulation. Typically, insulators, such as plastics, have dielectric strength values of about 100V per micron or more. As a high dielectric constant reduces the field within the internal insulator 310, a combination of a high dielectric constant and a high dielectric strength gives a significant advantage. This can be achieved by using a single material that has the desired properties or it can be achieved by a double layer with the correct parameters and thickness. In addition, to further decreasing the possibility that the insulating layer 310 will fail, all sharp edges of the insulating layer 310 should be eliminated as by rounding the corners, etc., as illustrated in FIG. 7D using conventional techniques.

FIG. 6 illustrates a second type of treatment using the isolects 230, namely electric field generation by internal isolects 230. A body to which the isolects 230 are implanted is generally indicated at 311 and includes a skin surface 313 and a tumor 315. In this embodiment, the isolects 230 can have the shape of plates, wires or other shapes that can be inserted subcutaneously or a deeper location within the body 311 so as to generate an appropriate field at the target area (tumor 315).

In order to avoid overheating of the treated tissues, a selection of materials and field parameters is needed. The isolects insulating material should have minimal dielectric losses at the frequency ranges to be used during the treatment process. This factor can be taken into consideration when choosing the particular frequencies for the treatment. The direct heating of the tissues will most likely be dominated by the heating due to current flow (given by the I*R product). In addition, the isolect (insulated electrode) 230 and its surroundings should be made of materials that facilitate heat losses and its general structure should also facilitate head losses, i.e., minimal structures that block heat dissipation to the surroundings (air) as well as high heat conductivity. Using larger electrodes also minimizes the local sensation of heating, since it spreads the energy that is being transferred into the patient over a larger surface area. Preferably, the heating is minimized to the point where the patient's skin temperature never exceeds about 39° C.

Another way to reduce heating is to apply the field to the tissue being treated intermittently, by applying a field with a duty cycle between about 20% and about 50% instead of using a continuous field. For example, to achieve a duty cycle of 33%, the field would be repetitively switched on for one second, then switched off for two seconds. Preliminary experiments have shown that the efficacy of treatment using a field with a 33% duty cycle is roughly the same as for a field with a duty cycle of 100%. In alternative embodiments, the field could be switched on for one hour then switched off for one hour to achieve a duty cycle of 50%. Of course, switching at a rate of once per hour would not help minimize short-term heating. On the other hand, it could provide the patient with a welcome break from treatment.

It will also be appreciated that the present apparatus can further include a device for rotating the TTFields relative to the living tissue. For example and according to one embodiment, the alternating electric potential applies to the tissue being treated is rotated relative to the tissue using conventional devices, such as a mechanical device that upon activation, rotates various components of the present system.

The TTFields may be applied to different pairs of the insulated electrodes 230 in a consecutive manner in order to vary the direction of the TTFields that travel through the target region, as described in published application US2005/0209642, which is incorporated herein by reference. The changing of the field's direction may be implemented in a stepwise manner or in a continuous manner, also as described in published application US2005/0209642.

As described in published application US2005/0209642, it can be advantageous to apply a distribution of different frequencies to the population. For example, experiments indicate that using two frequencies of 170 kHz and 250 kHz to destroy a population of glioma cells would be more effective than using a single frequency of 200 kHz. When more than one frequency is used, the various frequencies may be applied sequentially in time. For example, in the case of glioma, field frequencies of 100, 150, 170, 200, 250, and 300 kHz may be applied during the first, second, third, fourth, fifth, and sixth minutes of treatment, respectively. That cycle of frequencies would then repeat during each successive six minutes of treatment. Alternatively, the frequency of the field may be swept in a stepless manner from 100 to 300 kHz. Optionally, this frequency cycling may be combined with the directional changes described above.

In an alternative embodiment, a signal that contains two or more frequencies components simultaneously (e.g., 170 kHz and 250 kHz) is applied to the electrodes to treat a population of cells that have a distribution of sizes. The various signals will add by superposition to create a field that includes all of the applied frequency components.

### EXAMPLE

The efficacy of the combined treatment of TTFields and depatuxizumab mafodotin was tested using two human glioma cell lines: U87-MG (ATCC) and U87MGde2-7 (Ludwig Institute for Cancer Research). All cells were grown in a humidified incubator supplied with 5% CO₂. U-87MGde2-7 and U87-MG were maintained in DMEM (Dulbecco's Modified Eagle's Medium) with high glucose, supplemented with 10% FBS (fetal bovine serum) and 1 mmol/L sodium pyruvate. U87MGde2-7 cells were maintained under selection with 400 mg/mL geneticin.

Cytotoxicity Assay: TTFields (1.75 V/cm RMS, 200 kHz) were applied for 72 hours using the inovitro system (as described in Giladi M, Schneiderman R S, Voloshin T, Porat Y, Munster M, Blat R, et al., Mitotic Spindle Disruption by Alternating Electric Fields Leads to Improper Chromosome Segregation and Mitotic Catastrophe in Cancer Cells. Sci Rep. 2015; 5:18046). The inovitro system is comprised of a TTFields generator and base plate containing 8 ceramic dishes per plate. The orientation of the TTFields was switched 90° every 1 second, thus covering the majority of the orientation axis of cell divisions, as described by Kirson et al. Kirson E D, Dbaly V, Tovarys F, Vymazal J, Soustiel J F, Itzhaki A, et al., Alternating electric fields arrest cell proliferation in animal tumor models and human brain tumors. Proc Natl Acad Sci USA 2007; 104(24):10152-7. Glioma cells were plated on a 22 mm round cover slip placed inside the inovitro dish. Following overnight incubation, the dishes were filled with 2 ml media containing depatuxizumab mafodotin or the isotype control concentrations of 0.01-100 nmol/L in 2 fold dilutions.

At the end of treatment, inhibition of tumor cell growth was analyzed quantitatively based on cell count performed using the EC800 flow cytometer (Sony Biotechnology, Japan).

Flow Cytometry: For detection of apoptosis, cells were double stained with FITC-conjugated Annexin V (MEBCYTO 4700 Apoptosis Kit; MBL) and 7-Aminoactinomycin D (7-AAD; Biolegend) as per manufacturer's instructions. Data acquisition was obtained using iCyt EC800 (Sony Biotechnology) flow cytometer. Fluorescence signals were collected at the wavelengths of 525/50 nm for Annexin V and 665/30 nm for 7-AAD. The data was analyzed using the Flowjo software (TreeStar).

Statistical Analysis: Data are expressed as mean SD, and the statistical significance of differences was assessed using GraphPad Prism 6 software (GraphPad Software, La Jolla, Calif.). Differences between all groups were compared with each other, and were considered significant at values of 0.05>*p>0.01, **p<0.01, and ***p<0.001. All experiments were repeated at least three times.

Results

Efficacy of TTFields and Depatuxizumab Mafodotin on U-87MG Cells

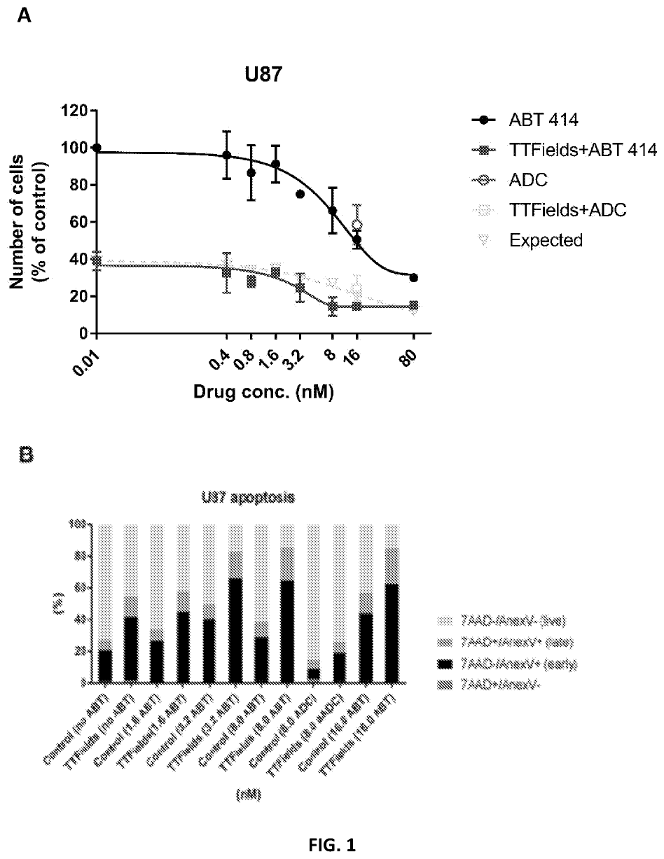

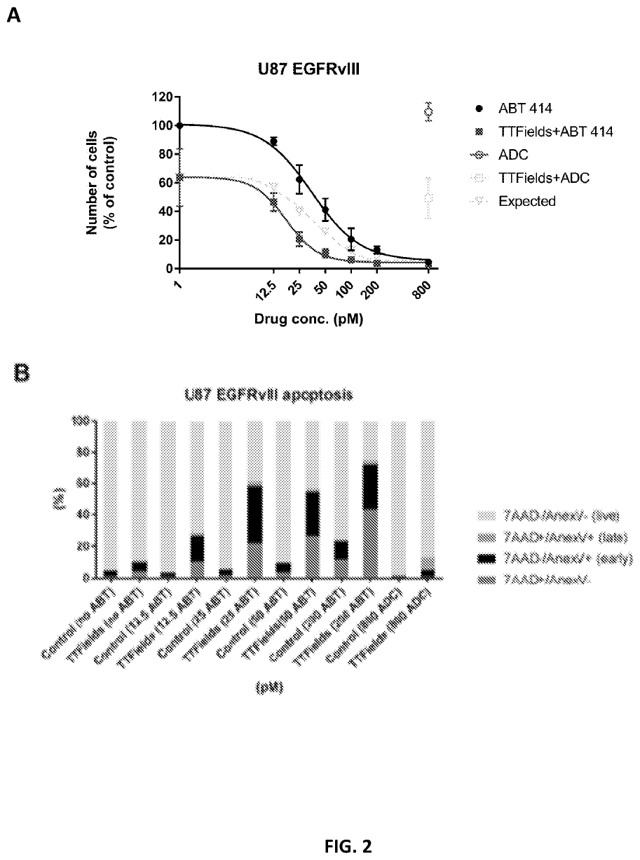

Titration experiments showed that the effective dose of depatuxizumab mafodotin needed to inhibit growth of the U87-MG cells was within the nM range. Some variation from the previously reported IC50 values were observed and those were mainly attributed to the difference in the enumeration technique between this study (cell count) and the previous research (luminescence based assay). As shown in FIG. 1A, the combined treatment of TTFields and depatuxizumab mafodotin (which is referred to in FIGS. 1-2 as either “ABT-414” or “ABT”) led to a significant reduction in the number of U87-MG cells (P<0.001) as compared to each treatment alone at all drug concentrations except 80 nm. In contrast, the combination of TTfields and a control ADC, Ab095-MMAF ADC (i.e., Ab095, an antibody that targets tetanus toxoid, conjugated to MMAF to form a non-specific ADC that is referred to in FIGS. 1-2 as “ADC”, see also Larrick et al., 1992, Immunological Reviews, 69-85), led to a non-significant reduction in the number of cells as compared to TTFields treatment alone. A rapid increase in the number of cells undergoing apoptosis (both early and late) was seen for the combination treatment of TTfields and depatuxizumab mafodotin in all tested concentrations (as compared to either of those treatments taken alone), as shown in FIG. 1B. In contrast, the percentage of apoptotic cells following treatment with Ab095-MMAF alone was similar to the percentage for Ab095-MMAF ADC combined with TTFields.

Efficacy of TTFields and Depatuxizumab Mafodotin on U87MGde2-7 Cells

Titration experiment revealed that depatuxizumab mafodotin effectivity on U87MGde2-7 cells is within the pM range. The combined treatment of TTFields and depatuxizumab mafodotin led to a significant reduction in the number of U87MGde2-7 cells compared to either treatment alone, as shown in FIG. 2A. In contrast, the combined treatment of TTFields and Ab095-MMAF led to a non-significant reduction in the number of cells as compared to TTFields treatment alone. As in the case of U-87 MG, a rapid increase in the number of cells undergoing apoptosis (both early and late) was seen for the combination treatment of TTFields and depatuxizumab mafodotin in all tested concentrations, as shown in FIG. 2B. In contrast, there was no increase in the percentage of apoptotic cells following treatment with Ab095-MMAF or TTFields+Ab095-MMAF as compared to control cultures or to cells treated with TTFields alone.

These results demonstrate that in U87 cells, the combination of TTFields and depatuxizumab mafodotin in the nM range led to a significant reduction in cell number and to an increase in apoptosis as compared to each treatment alone. In U87EGFRvIII cells, the combined treatment of TTFields and depatuxizumab mafodotin in the pM range led to a significant reduction in cell number and to an increase in apoptosis as compared to each treatment alone. TTfields application had very little effect on cells treated with the Ab095 MMAF non-specific ADC.

ANTIBODY SEQUENCE TABLE

| SEQ | | |
|---|---|---|
| ID NO | Description | Sequence |
| 1 | Anti-EGFR Antibody | QVQLQESGPGLVKPSQTLSLTCTVSGYSISSDFAWN |
| | Heavy Chain | WIRQPPGKGLEVVMGYISYSGNTRYQPSLKSRITIS |
| | | RDTSKNQFFLKLNSVTAADTATYYCVTAGRGFPYWG |
| | | QGTLVTVSSASTKGPSVFPLAPSSKSTSGGTAALGC |
| | | LVKDYFPEPVTVSWNSGALTSGVHTFPAVLQSSGLY |
| | | SLSSVVTVPSSSLGTQTYICNVNHKPSNTKVDKKVE |
| | | PKSCDKTHTCPPCPAPELLGGPSVFLFPPKPKDTLM |
| | | ISRTPEVTCVVVDVSHEDPEVKFNWYVDGVEVHNAK |
| | | TKPREEQYNSTYRVVSVLTVLHQDWLNGKEYKCKVS |
| | | NKALPAPIEKTISKAKGQPREPQVYTLPPSRDELTK |
| | | NQVSLTCLVKGFYPSDIAVEWESNGQPENNYKTTPP |
| | | VLDSDGSFFLYSKLTVDKSRWQQGNVFSCSVMHEAL |
| | | HNHYTQKSLSLSPGK |
| 2 | Anti-EGFR Antibody | QVQLQESGPGLVKPSQTLSLTCTVSGYSISSDFAWN |
| | Heavy Chain Variable | WIRQPPGKGLEWMGYISYSGNTRYQPSLKSRITISR |
| | Region | DTSKNQFFLKLNSVTAADTATYYCVTAGRGFPYWGQ |
| | | GTLVTVSS |
| 3 | Anti-EGFR Antibody | SDFAWN |
| | HC CDR1 | |
| 4 | Anti-EGFR Antibody | YISYSGNTRYQPSLKS |
| | HC CDR2 | |
| 5 | Anti-EGFR Antibody | VTAGRGFPY |
| | HC CDR3 | |
| 6 | Anti-EGFR Antibody | DIQMTQSPSSMSVSVGDRVTITCHSSQDINSNIGW |
| | light chain (LC) | LQQKPGKSFKGLIYHGTNLDDGVPSRFSGSGSGTD |
| | | YTLTISSLQPEDFATYYCVQYAQFPWTFGGGTKLE |
| | | IKRTVAAPSVFIFPPSDEQLKSGTASVVCLLNNFY |
| | | PREAKVQWKVDNALQSGNSQESVTEQDSKDSTYSL |
| | | SSTLTLSKADYEKHKVYACEVTHQGLSSPVTKSFN |
| | | RGEC |
| 7 | Anti-EGFR Antibody | DIQMTQSPSSMSVSVGDRVTITCHSSQDINSNIGW |
| | Light Chain | LQQKPGKSFKGLIYHGTNLDDGVPSRFSGSGSGTD |
| | Variable Region | YTLTISSLQPEDFATYYCVQYAQFPWTFGGGTKLE |
| | | IK |
| 8 | Anti-EGFR Antibody | HSSQDINSNIG |
| | LC CDR1 | |
| 9 | Anti-EGFR Antibody | HGTNLDD |
| | LC CDR2 | |
| 10 | Anti-EGFR Antibody | VQYAQFPWT |
| | LC CDR3 | |

What is claimed is:

1. A method for treating cancer in a patient harboring an EGFR-expressing tumor, the method comprising
(i) applying an AC electric field to a target area, wherein the target area comprises an EGFR-expressing tumor or cancer cell); and
(ii) administering an effective amount of depatuxizumab mafodotin.

2. The method according to claim 1, wherein the cancer expresses the mutant EGFRvIII.

3. The method according to claim 1, wherein the cancer is glioblastoma.

4. The method according to claim 3, wherein the electric field has a frequency between 50 kHz and 500 kHz.

5. The method according to claim 4, wherein the electric field has a frequency of 100 kHz to 300 kHz.

6. The method according to claim 5, wherein the electric field has a frequency of about 200 kHz.

7. The method according to claim 4, wherein the strength of the electric field in at least a portion of the target region is between about 1 V/cm and about 5 V/cm.

8. The method according to claim 4, wherein at least two different frequencies are imposed in the target region.

9. The method according to claim 5, wherein at least two different frequencies are imposed in the target region.

10. A method for inhibiting tumor growth, the method comprising:
(i) applying an AC electric field to a target area, wherein the target area comprises an EGFR-expressing tumor or cancer cell) and
(ii) administering an effective amount of depatuxizumab mafodotin.
